(19) United States
(12) Patent Application Publication
(10) Pub. No.: US 2014/0052023 A1
(43) Pub. Date: Feb. 20, 2014

(54) Single Use Lancet Assembly

(71) Applicant: Charles D. Starnes, Coral Springs, FL (US)

(72) Inventor: Charles D. Starnes, Coral Springs, FL (US)

(21) Appl. No.: 13/586,207

(22) Filed: Aug. 15, 2012

(51) Int. Cl. A61B 5/151

(52) U.S. Cl. CPC A61B 5/15144; A61B 5/15117; A61B 5/15111; A61B 5/1513
USPC 600/583

(57) ABSTRACT

A single use, spring-driven lancet assembly having a housing, a removable cap, a lancet, a lancet trigger member and a spring. The lancet trigger comprises a nose that extends from the front of the housing. When the lancet assembly is pressed against the skin or vice versa, the trigger is pushed toward the rear of the housing. A pair of flexible trigger arm members in contact with cocking lugs on the lancet body push the lancet to the rear and compress the spring. As the trigger and lancet move to the rear within the housing, the flexible arm members of the trigger are spread apart and the released lancet is propelled forward. The spring recoils into a neutral position and draws the lancet and its needle back into the housing. The lancet assembly cannot be re-cocked and the lancet needle cannot be intentionally or accidentally extended from the housing.

### BACKGROUND OF THE INVENTION

The invention relates generally to the field of devices or assemblies incorporating lancets, needles or the like used to pierce or prick the skin in order to draw a sample of blood or create a small droplet of blood on the surface of the skin. More particularly, the invention relates to such devices or assemblies that utilize a spring to propel the lancet a short distance out of a protective housing and then retract the lancet into the housing. Even more particularly, the invention relates to such devices or assemblies that are structured to preclude re-use of the device or assembly.

Spring operated, skin piercing lancet assemblies are well known in the art. Examples of such assemblies, some of which comprise structure to preclude re-use of the lancet assembly, are shown for example in U.S. Pat. No. 4,817,603 to Turner et al., U.S. Pat. No. 5,439,473 to Jorgensen, U.S. Pat. No. 5,628,765 to Morita, U.S. Pat. No. 5,643,306 to Schraga, U.S. Pat. No. 5,755,733 to Morita, U.S. Pat. No. 5,908,434 to Schraga, U.S. Pat. No. 6,053,930 to Ruppert, U.S. Pat. No. 6,149,608 to Marshall et al., U.S. Pat. No. 6,168,606 to Levin et al., U.S. Pat. No. 6,258,112 to Schraga, U.S. Pat. No. 6,432,120 to Teo, U.S. Pat. No. 6,514,270 to Schraga, U.S. Pat. No. 6,764,496 to Schraga, U.S. Pat. No. 6,540,763 to Teo at al., U.S. Pat. No. 6,918,918 to Schraga, U.S. Pat. No. 7,322,997 to Shi, U.S. Pat. No. 7,775,991 to Feaster et al., and U.S. Pat. No. 7,785,338 to Kuhr et al.

To prevent the accidental transmittal of disease that may occur from multiple usages of a lancet assembly and to prevent accidental pricking or the transmittal of disease to post-use handlers of the lancet assemblies, assemblies that provide a mechanism for retaining the lancet in a recessed condition within the housing and which preclude any extension, accidental or intentional, of the lancet from the housing after its use are preferred. It is an object of this invention to provide structural embodiments and designs for a spring operated, single use, lancet assembly that precludes re-use or re-extension of the lancet and addresses problems or shortcomings inherent in the prior art.

### SUMMARY OF THE INVENTION

The invention is a single use lancet assembly of the type comprising a spring driven lancet that is retracted into a recessed position within a protective housing after the lancet has pierced the skin, and further wherein after its use the lancet cannot be extended from the housing. The lancet assembly comprises in general a housing, a removable cap, a lancet, a lancet trigger member and a spring. Removal of the cap simultaneously removes a needle cover to expose the needle for use. The lancet trigger comprises a nose that extends from the front of the housing. When the lancet assembly is pressed against the skin, or vice versa, the trigger is pushed toward the rear of the housing. A pair of flexible trigger arm members having cocking flanges in contact with cocking lugs on the lancet body push the lancet to the rear and compress the spring. As the trigger and lancet move to the rear within the housing, the flexible arm members of the trigger are spread apart until the separation distance is such that the lancet cocking lugs are released and the lancet is propelled forward by the spring, the needle of the lancet extending beyond the trigger nose to pierce the skin. The spring is attached to the lancet such that after extension the spring recoils into a neutral position and draws the lancet and its needle fully back into the housing. The trigger arm members are restrained from subsequent forward movement by detention shoulders such that the trigger cocking flanges remain to the rear of the lancet cocking lugs and the lancet assembly cannot be re-cocked and re-used, and further such that the lancet needle cannot be intentionally or accidentally extended from the housing.

In other terms, the invention can be summarized as a lancet assembly comprising a housing comprising a proximal end, a distal end, a distal opening, an angled guide surface and a detention shoulder; a cap removably mounted onto said housing and covering said distal opening; a trigger member positioned within said housing, said trigger member comprising a nose extending through said distal opening of said housing, a body, a lancet bore extending through said nose and said body, an arm member connected to said body and extending toward said proximal end of said housing, said arm member having a free end, a detention member mounted on said free end of said arm member, and a cocking flange positioned on said arm member; a lancet positioned within said housing and extendable through said lancet bore of said trigger member and through said distal opening of said housing, said lancet comprising a main body, a needle extending from said main body, and a cocking lug; a spring member connected to said lancet; wherein said trigger member and said lancet are retained within said housing in a manner allowing movement of said trigger member toward said proximal end of said housing and allowing movement of said lancet toward said proximal end and said distal end of said housing; wherein in a pre-cocked condition said needle is positioned within said housing, and said cocking flange of said trigger member is positioned closer to said distal end of said housing than said cocking lug of said lancet; wherein with said cap removed from said housing said trigger member may be moved toward said proximal end of said housing such that said cocking flange abuts said cocking lug, thereby moving said lancet main body toward said proximal end of said housing and loading said spring, such that said free end of said arm member is biased outwardly by said angled guide surface as said trigger member is moved toward said proximal end of said housing, such that said cocking flange is moved out of contact with said cocking lug, thereby allowing said spring to propel said lancet needle through and beyond said distal opening of said housing, and such that said detention member of said arm member is restrained by said detention shoulder of said angled guide surface, thereby preventing movement of said trigger member toward said distal end of said housing; wherein after extension of said needle beyond said housing distal opening said spring retracts said needle into said housing, said cocking lug of said lancet now being positioned closer to the distal end of said housing than said cocking flange of said trigger member, such that said trigger member is now incapable of moving said lancet axially toward said proximal end of said housing. Furthermore, in various combinations the invention may be described wherein said housing comprises a pair of angled guide surfaces and detention shoulders, and wherein said trigger member comprises a pair of arm members and detention members, wherein said lancet comprises a pair of cocking lugs and said trigger member comprises a pair of cocking flanges, and further comprising a needle cover removably connected to said lancet main body and covering said needle, wherein said lancet needle cover is retained by said cap such that removal of said cap from said housing results in removal of said lancet needle cover from said lancet main body.

Alternatively, the lancet assembly may be summarized as comprising a housing comprising a proximal end, a distal end, a distal opening, an angled guide surface and a detention shoulder; a cap removably mounted onto said housing and covering said distal opening; a lancet retained within said housing, said lancet comprising a main body, a needle extending from said main body, and a cocking lug, said lancet comprising a longitudinal axis, wherein said lancet is movable in both axial directions relative to said housing; a trigger member positioned within said housing, said trigger member comprising a nose extending through said distal opening of said housing, a body, a lancet bore extending through said nose and said body, an arm member connected to said body and extending toward said proximal end of said housing, said arm member having a free end, a detention member mounted on said free end of said arm member, and a cocking flange positioned on said arm member, wherein said trigger member is movable in the axial direction toward said proximal end of said housing; said lancet being disposed within said lancet bore of said trigger member; a spring member connected to said lancet; wherein in a pre-cocked condition said needle is positioned within said housing, and said cocking flange of said trigger member is positioned closer to said distal end of said housing than said cocking lug of said lancet; wherein with said cap removed from said housing said trigger member may be moved axially toward said proximal end of said housing such that said cocking flange abuts said cocking lug, thereby moving said lancet main body axially toward said proximal end of said housing and loading said spring, such that said free end of said arm member is biased outwardly by said angled guide surface as said trigger member is moved toward said proximal end of said housing, such that said cocking flange is moved out of contact with said cocking lug, thereby allowing said spring to propel said lancet needle axially through and beyond said distal opening of said housing, and such that said detention member of said arm member is restrained by said detention shoulder of said angled guide surface, thereby preventing axial movement of said trigger member toward said distal end of said housing; wherein after extension of said needle beyond said housing distal opening said spring axially retracts said needle into said housing, said cocking lug of said lancet now being positioned closer to the distal end of said housing than said cocking flange of said trigger member, such that said trigger member is rendered incapable of moving said lancet in the axial direction toward said proximal end of said housing. Furthermore, in various combinations the lancet assembly may be described wherein said housing comprises a pair of angled guide surfaces and detention shoulders, and wherein said trigger member comprises a pair of arm members and detention members; wherein said lancet comprises a pair of cocking lugs and said trigger member comprises a pair of cocking flanges, and further comprising a needle cover removably connected to said main body and covering said needle, wherein said lancet needle cover is retained by said cap such that removal of said cap from said housing results in removal of said lancet needle cover from said lancet main body.

Still further alternatively, a lancet assembly comprising a housing comprising a proximal end, a distal end, a distal opening, a pair of angled guide surfaces and a pair of detention shoulders; a cap removably mounted onto said housing and covering said distal opening; a trigger member positioned within said housing, said trigger member comprising a nose extending through said distal opening of said housing, a body, a lancet bore extending through said nose and said body, a pair of arm members connected to said body and extending toward said proximal end of said housing, said arm members each having a free end, a detention member mounted on each said free end of said arm members, and a cocking flange positioned on each said arm member; a lancet positioned within said housing and extending through said lancet bore, said lancet comprising a main body, a needle extending from said main body, and a pair of cocking lugs; and a spring member connected to said lancet and to said housing. And further in various combinations wherein said detention members abut said angled guide surfaces and said cocking flanges abut said cocking lugs, further comprising a needle cover removably connected to said lancet main body and covering said needle, wherein said needle cover is connected to said cap in a manner such that rotating said cap relative to said housing rotates said needle cover and separates said needle cover from said lancet main body, wherein said trigger member and said lancet are disposed within said housing such that said trigger member is movable toward the proximal end of said housing and said lancet is movable toward said proximal end and said distal end of said housing, wherein movement of said trigger member toward said proximal end of said housing results in movement of said lancet first toward proximal end of said housing and then toward said distal end, wherein after said lancet moves toward said distal end of said housing, said spring retracts said lancet needle into said housing by moving said lancet toward the proximal end of said housing, wherein said trigger member, said cocking flanges, said cocking lugs, said angled guide surfaces and said arm members are disposed within said housing such that movement of said trigger member toward said proximal end of said housing results in separation of said arm members such that said cocking flanges no longer abut said cocking lugs, thereby allowing said lancet to move toward said distal end of said housing, wherein said detention members and said detention shoulders are disposed within said housing such that said movement of said trigger member toward said proximal end of said housing results in said detention members abutting said detention shoulders, thereby preventing said trigger member from moving toward said distal end of said housing.

### BRIEF DESCRIPTION OF THE DRAWINGS

FIG. 1 is a perspective view of an embodiment of the single use lancet assembly showing the device in the pre-cocked condition with the cap removed.

FIG. 2 is a perspective view of an embodiment of the cap for the single use lancet assembly.

FIG. 3 is a perspective exterior view of an embodiment of the top half of the housing of the single use lancet assembly.

FIG. 4 is a perspective interior view of the top half of the housing of FIG. 3.

FIG. 5 is a perspective view of the exterior an embodiment of the bottom half of the housing of the single use lancet assembly.

FIG. 6 is a perspective view of the interior of the bottom half of the housing of FIG. 5.

FIG. 7 is a perspective view of an embodiment of the lancet prior to removal of the needle cover.

FIG. 8 is a perspective view of the lancet of FIG. 7 with the needle cover removed.

FIG. 9 is a top view of the lancet of FIG. 7.

FIG. 10 is a side view of the lancet of FIG. 7.

FIG. 11 is a top perspective view of an embodiment of the trigger member.

FIG. 12 is a bottom perspective view of the trigger member of FIG. 11.

FIG. 13 is a top view of the trigger member of FIG. 11.

FIG. 14 is a side view of the trigger member of FIG. 11.

FIG. 15 is an end view of the trigger member of FIG. 11.

FIG. 16 is a top view of the interior of the bottom half of the housing of FIG. 5.

FIG. 17 is a top view, partially exposed and partially in cross-section, of the single use lancet assembly in the pre-use condition prior to removal of the cap and needle cover from the housing and lancet, respectively.

FIG. 18 is top view similar to FIG. 17 but with the cap and needle cover removed and showing the single use lancet assembly in the fired and extended piercing condition.

FIG. 19 is a top view similar to FIG. 18 showing the single use lancet assembly in the post-firing retracted and recessed condition.

### DETAILED DESCRIPTION OF THE INVENTION

With reference to the drawings, the invention will now be described in detail with regard for the best mode and preferred embodiment. In a broad sense, the invention is a single use lancet assembly of the type comprising a spring driven lancet that is retracted into a recessed position within a protective housing after the lancet has pierced the skin, and further wherein after its use the lancet cannot subsequently be extended from the housing. The lancet assembly comprises a main body or housing 11 that is gripped by the user during operation of the device, a removable cap member 20 that is removed by the user prior to commencing the skin piercing operation, a lancet 40 having a sharp point, blade or needle 42 adapted to easily pierce the skin such that a drop or small amount of blood will be exposed on the surface of the skin, a spring member 60 to propel and then retract the lancet 40, and a trigger member 50 that cocks or arms the lancet assembly and then releases or fires the lancet 40 to pierce the skin, the lancet assembly being structured such that after the lancet 40 has been fired it cannot be re-cocked or manually extended from the housing 11.

For purposes of the disclosure, the term distal shall refer herein to the front or forward direction, i.e., the direction toward the skin when the device is in use, and the term proximal shall refer to the rear or rearward direction. The lancet shall define a longitudinal axis, and further such that the axial direction is the direction of travel of the lancet during the cocking and piercing operation. The term needle shall be used herein to refer to any suitable lancet piercing member.

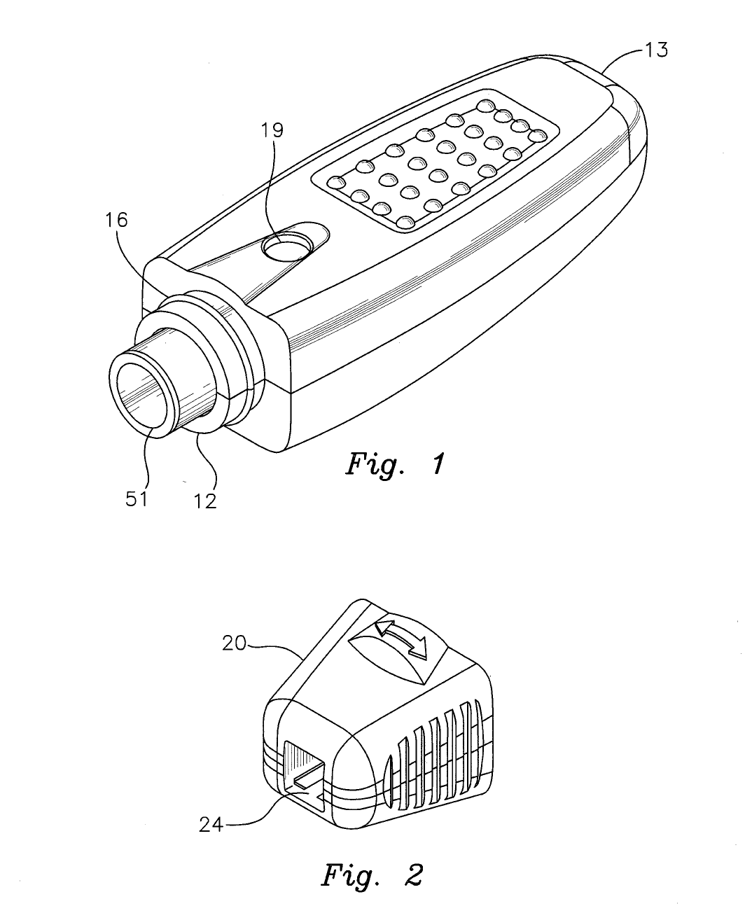

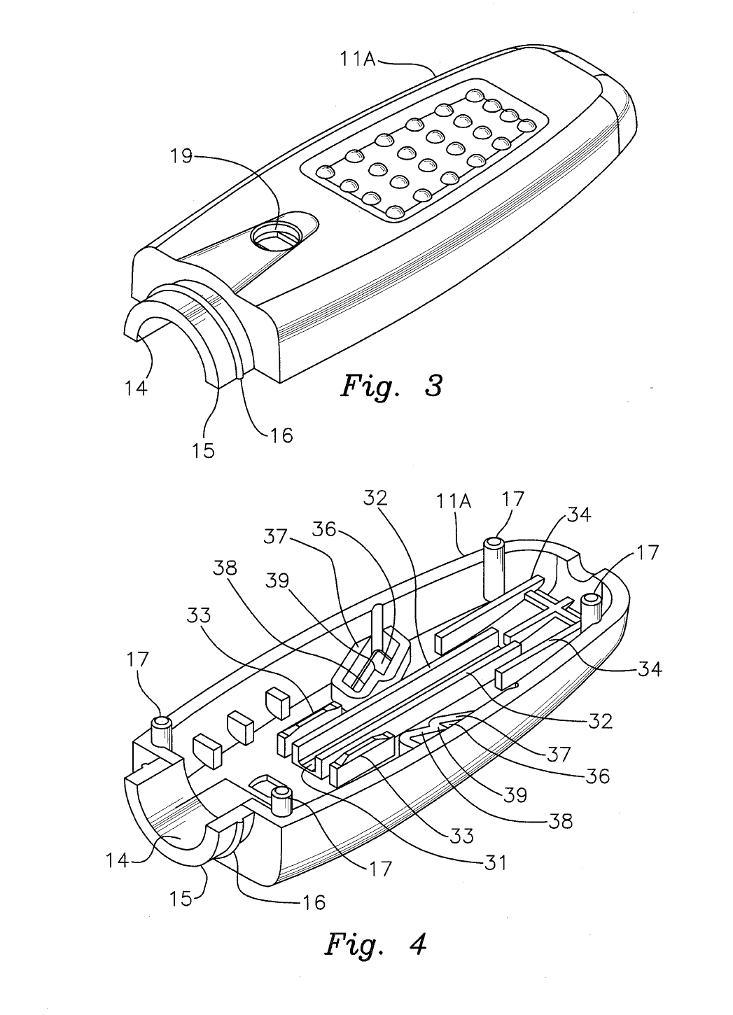

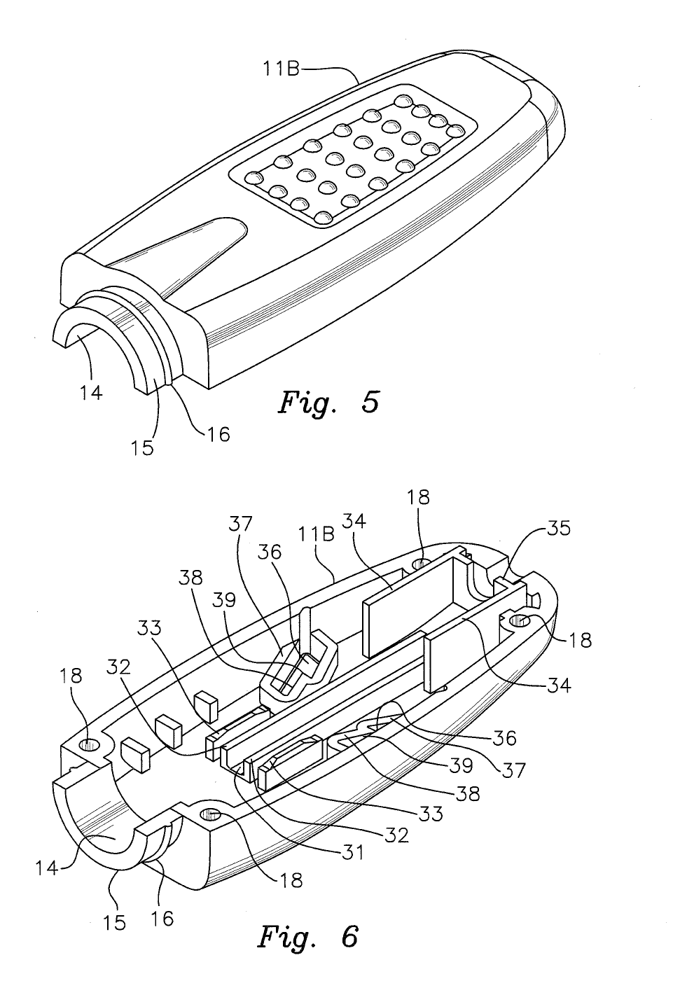

FIGS. 1 and 3 through 6 illustrate an embodiment for the housing 11, the drawings showing the exterior and the interior. The housing 11 is preferably comprised of two components or halves, which for ease of reference are designated as a top half 11A and a bottom half 11B, as this construction is preferable for manufacture. The housing top half 11A and housing bottom half 11B are joined in known manner after the lancet assembly has been assembled, such as with adhesives, melt bonding, friction fitting or the like. As shown in the drawings, alignment members such as mating posts 17 and mating bores 18 to receive the mating posts 17 may be provided. In the assembled form the housing 11 defines a distal end 12 and a proximal end 13, with the distal end 12 being the forward or front end of the lancet assembly that is directed or placed against the skin during the piercing operation and with the proximal end 13 being the opposite end that is the rear or back end of the device. A neck member 15 having a distal opening or bore 14 is provided on the distal end of the housing 11, the distal bore 14 being adapted to coaxially receive the trigger nose 51 and the lancet 40 in a manner such that the trigger nose 51 and the lancet 40 may move axially relative to the housing 11, the axial direction being taken herein to be that of the longitudinal axis of the lancet 40. The distal bore 14 is not required to have any particular cross-sectional shape, and as such may be tubular, polygonal, a combination thereof, etc. The housing 11 may be provided with a status window 19 open to the interior of the housing 11 to provide a visual indication of whether or not the lancet assembly has been used.

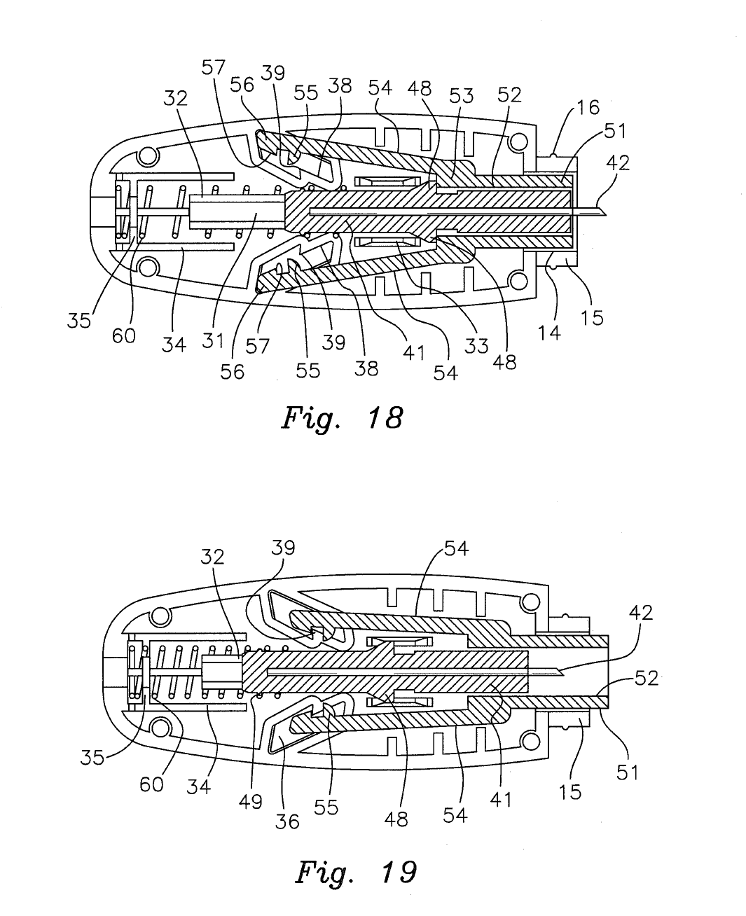

The lancet assembly further comprises a removable cap member 20, as shown in FIG. 2. The cap member 20 temporarily mounts on the distal end 12 of the housing 11 and the cap open interior 21 receives and covers the exposed trigger nose 51 prior to use. The cap member 20 may be mounted and retained in known manner, and in the embodiment shown is retained by a generally annular retention ring or ridge 16 disposed on the exterior of the housing neck member 15, the retention ring 16 being received by a retention channel 22 disposed within the cap member 20, as seen in FIG. 17. Alternatively, the cap member 20 may for example be retained on the housing 11 by screw threading, spaced detention lugs, etc. Preferably, the mounting mechanism allows or requires the cap member 20 to be rotated relative to the housing 11 prior to removal in order to simultaneously detach a needle cover member 43 from the needle 42, such that the needle cover 43 is removed from the lancet assembly and the needle 42 is in condition for use when the cap member 20 is removed, as explained in greater detail below. The cap member 20 may be provided with enhanced gripping surfaces, physical gripping members, directional arrows, etc.

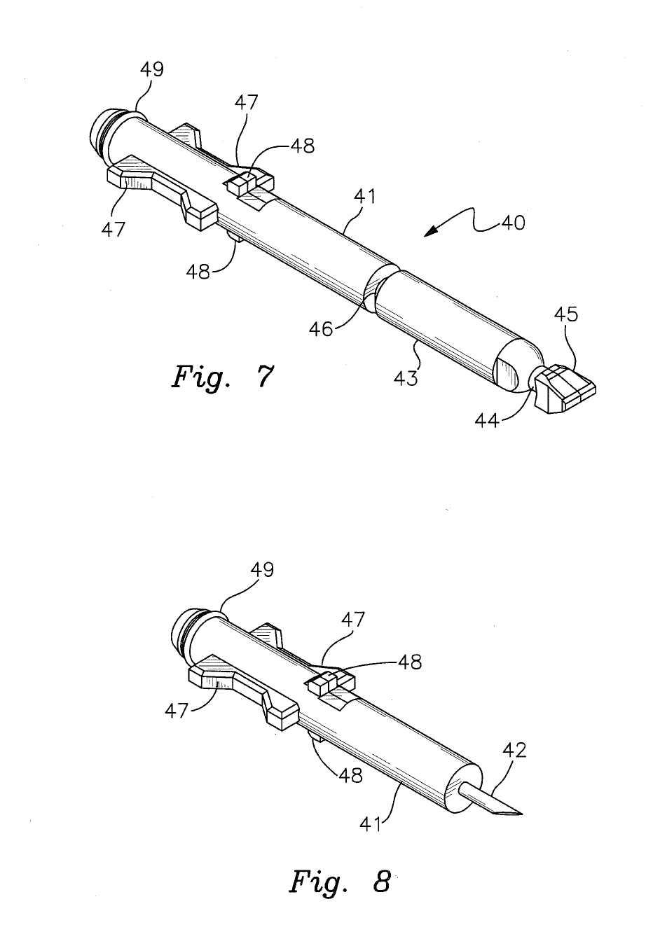

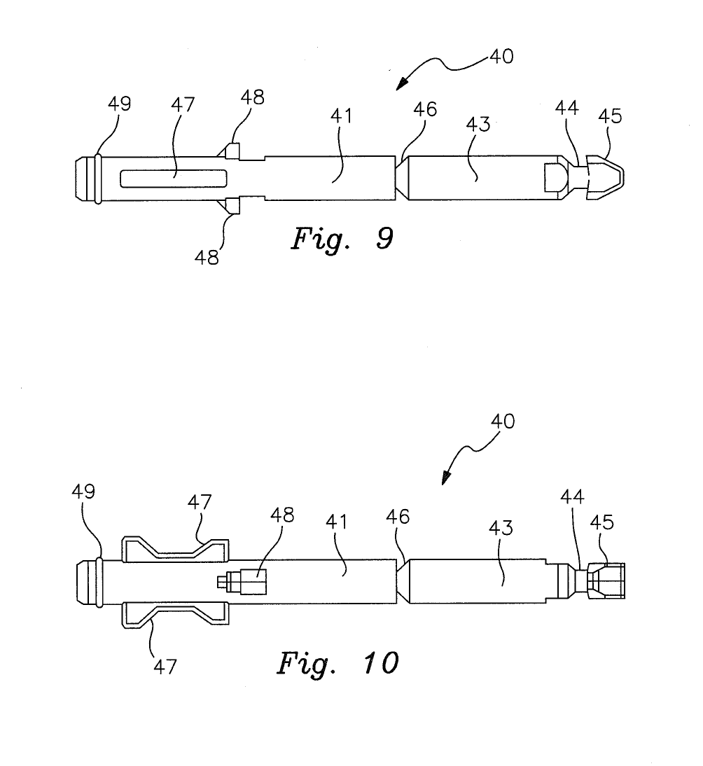

The lancet 40, an embodiment of which is shown in FIGS. 7 through 10, comprises a sharp point, blade or needle 42 (the term needle being used herein to refer to any such piercing member) extending coaxially from the distal end of an elongated, preferably cylindrical, main body 41, and an elongated needle cover 43, also preferably cylindrical, attached coaxially to and extending from the distal end of the main body 41 and covering or encasing the needle 42. The needle cover 43 is joined to the main body 41 by a reduced or necked portion 46, such that a relatively small amount of plastic material forms the juncture between the main body 41 and the needle cover 43. In this manner, the juncture is frangible and the needle cover 43 is easily separated from the lancet main body 41 by rotation of the needle cover 43 relative to the main body 41. The length of the needle cover 43 is such that the distal end of the needle cover 43 extends beyond the distal end 12 of the housing 11 and preferably beyond the distal end of the trigger nose 51. In a basic embodiment, with the cap member 20 removed from the housing 11, the exposed portion of the needle cover 43 is grasped and twisted for removal from the lancet main body 41 and to expose the needle 42 for later use.

In a preferred embodiment the lancet needle cover 43 is retained by the cap member 20 in a mechanically interlocking manner such that removal of the cap member 20 simultaneously results in separation and removal of the needle cover 43 from the lancet main body 41. The distal end of the lancet needle cover 43 is provided with a cover head member 45 connected to the needle cover 43 by a narrow neck member 44. As seen in FIG. 17, the cap member 20 is provided with a grappling channel 24 having inwardly extending hook or grappling members 23. With the cap member 20 mounted onto the housing 11, the cover head member 45 is positioned within the grappling channel 24 such that the grappling members 23 are disposed to the rear of the cover head member 45. The grappling channel 24 and/or the grappling members 23 are structured such that rotation of the cap member 20 results in corresponding rotation of the cover head member 45 and the needle cover 43 about the longitudinal axis. For example, the grappling members 23 and/or the grappling channel 24 are structured to present corners, edges, abutment members or an overall polygonal profile that corresponds to the profile of the cover head 45. The lancet main body 41, disposed within housing 11, is precluded from rotation by at least one and preferably a pair of longitudinal guide flanges 47 extending outwardly from the main body 41, the guide flanges 47 being restricted from rotational movement about the longitudinal axis of the lancet 40 by channel walls 32 mounted on the interior of housing 11, the channel walls 32 defining a lancet guide channel 31 that receives the guide flanges 47 such that only linear movement of the lancet main body 41 in either axial direction is permitted. Thus, rotation of the cap member 20 breaks the junction between the lancet main body 41 and the needle cover 43, and removal of the cap member 20 simultaneously removes the needle cover 43 from the housing 11.

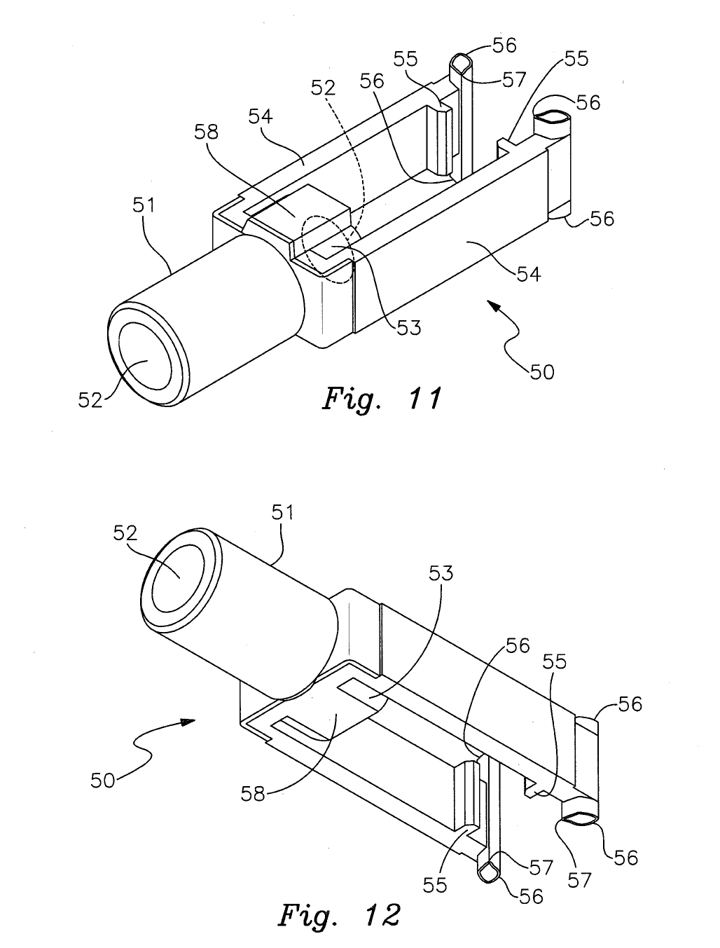

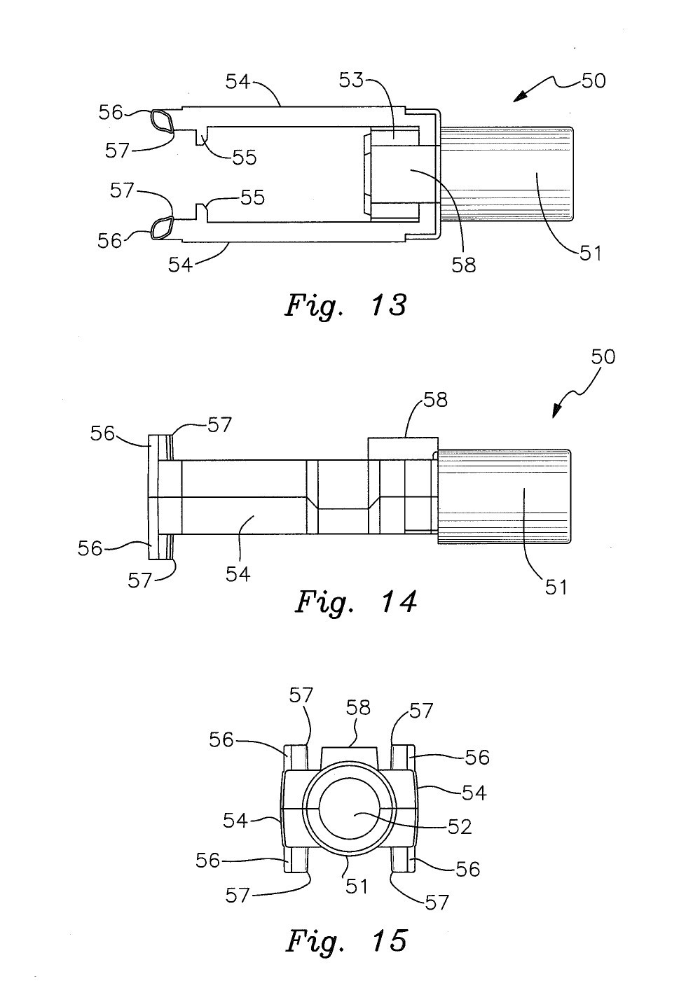

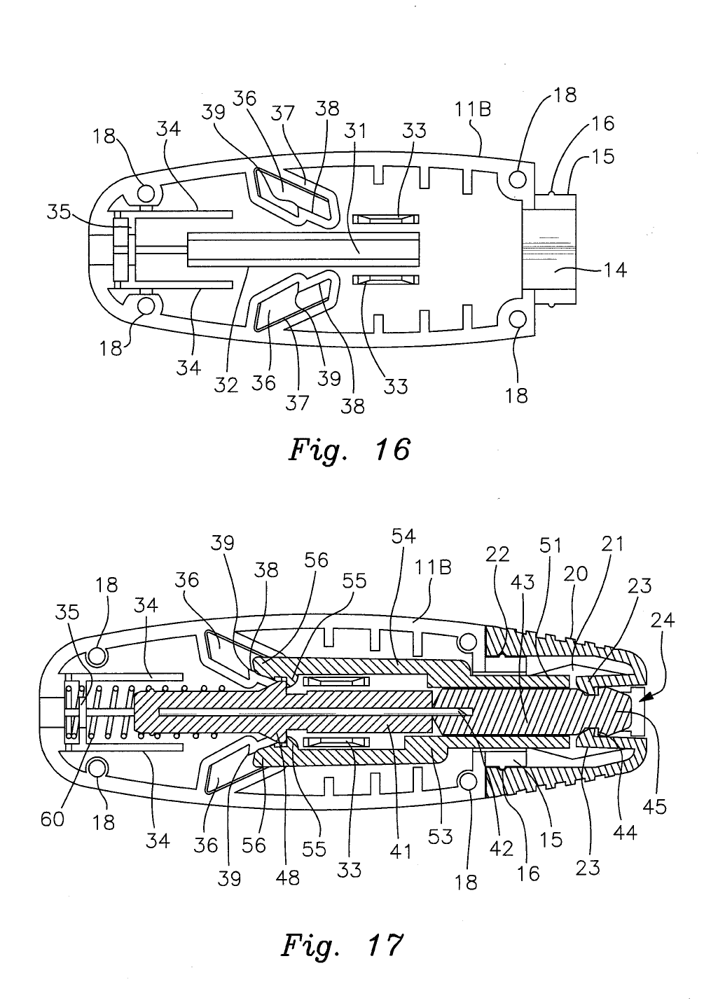

The trigger member 50, an embodiment of which is shown in FIGS. 11 through 15, is the element that controls and actuates the cocking and firing of the lancet 40. The trigger member 50 comprises a main body 53, a nose member 51 that extends from the main body 53 in the distal direction, and at least one and preferably a pair of opposing arm members 54. A lancet receiving bore 52 extends longitudinally through the nose member 51 and the main body 53, the trigger bore 52 being sized such that the lancet needle cover 43 is received therein in the pre-use condition, and further such that the distal portion of the lancet main body 41 moves freely in the distal and proximal directions within the trigger bore 52. The length of the trigger nose 51 is such that it extends through and beyond the housing neck member 15 when the lancet assembly is assembled.

The arm members 54 extend in the proximal direction from the trigger main body 53, the distal ends of the arm members 54 being connected to the main body 53 and the proximal ends of the arm members 54 being free, unconnected ends. The arm members 54 are composed of a relatively rigid yet slightly flexible and resilient material, preferably a plastic, whereby the proximal ends of the arm members 54 may be flexed or biased outwardly from a neutral position in which the arm members 54 reside generally parallel to the longitudinal axis of the lancet 40. The proximal free ends of the arm members 54 comprise post or detention members 56 that extend up or down, and preferably in both directions, from the arm members 54. Thus, the detention members 56 extend toward the housing top half 11A and toward the housing bottom half 11B. Preferably the detention members 56 further comprise an edged or cornered surface structure or catch structure 57, the catch structure 57 facing generally inward towards the lancet main body 40. Each arm member 54 is provided with an inwardly extending cocking flange member 55, the cocking flange members 55 adapted to abut at least one and preferably a pair of cocking lug members 48 extending laterally from the lancet main body 41. The lancet cocking lug members 48 are disposed orthogonally to the lancet guide flange members 47. The trigger member 40 may be provided with an indicator panel member 58 that is visible through the status window 19 of the housing 11 after the lancet assembly has been used.

A spring member 60, preferably of the helical type, is coaxially aligned with the lancet 40. The spring 60 is connected to the proximal end of the lancet main body 41, preferably such that a distal portion of the spring 60 encircles a proximal portion of the lancet main body 41. The spring 60 may be retained on the lancet main body 41 in any suitable manner, such as for example by providing an annular retention ring or shoulder member 49 on the lancet main body 41. Connection of the spring member 60 to the lancet main body 41 is necessary so that the lancet main body 41 and needle 42 will be retracted completely into the housing 11 after the lancet 40 has been fired and the spring 60 assumes its neutral position. The spring 60 is positioned between a pair of opposing spring retention walls 34 disposed on the interior of one or both of the housing top and bottom halves 11A and 11B. A spring retention flange member 35 is provided on one or both of the housing top and bottom halves 11A and 11B to retain the proximal end of the spring 60 in place when the spring 60 expands to propel the lancet main body 41 in the distal direction.

The interior surfaces of the housing top and bottom halves 11A and 11B are provided with inwardly extending structural members, as best seen in FIGS. 4, 6 and 16. These structural members restrain and guide the operational components of the lancet assembly. Longitudinally extending channel walls define a lancet guide channel 31, the lancet guide channel receiving the lancet guide flanges 47 such that only linear movement of the lancet 40 in the distal and proximal directions is allowed, and further such that rotation of the lancet main body 41 about the longitudinal axis is precluded. Longitudinally extending guide ridges 33 may be provided to restrain and guide the lancet cocking lug members 48. The mechanism for releasing or firing the lancet 40 and retaining the trigger member 50 within the housing 11 after the lancet 40 has been fired comprises the combination of an angled guide wall or surface 38 and a detention shoulder 39, preferably located on both the housing top half 11A and the housing bottom half 11B. A peripheral wall 37 may also be provided whereby the combination of the peripheral wall 37, the angled guide surface 38 and the detention shoulder 39 define a trigger guide recess 36. The angled guide surfaces 38 are arranged such that the proximal ends of the angled guide surfaces 38 are farther from the longitudinal axis than the distal ends. In other words, the distance between the proximal ends of the opposing angled guide surfaces 38 on the housing top half 11A is greater than the distance between the distal ends of the opposing angled guide surfaces 38 on housing top half 11A, and likewise for housing bottom half 11B. The angled guide surfaces 38 and the detention shoulders 39 extend sufficient distance from the interior walls of the housing top and bottom halves 11A and 11B so as to laterally abut the extended detention members 56 of the trigger arm members 54.

FIG. 17 illustrates the lancet assembly in the pre-use or neutral status or condition. The lancet 40 is disposed within the trigger member 50 and the spring member 60 is connected to the proximal end of the lancet main body 41. The distal end of the lancet needle cover 43 extends through and beyond the trigger nose 51 and the housing neck 15. The lancet needle cover 43 is joined to the lancet main body 41. The extended detention members 56 of the trigger arm members 54 are positioned within the trigger guide recess 36, the detention members 56 abutting the outer sides of the angled guide surfaces 38 at or near the distal ends of the angled guide surfaces 38. The trigger arm members 54 are in the neutral, i.e., non-biased, position such that the inwardly facing trigger cocking flanges 55 abut the lancet cocking lug members 48 and are positioned on the distal side of the lancet cocking lug members 48. The cap member 20 is mounted onto the housing 11 and the lancet cover head 45 is retained by the cap grappling members 23.

To use the lancet assembly, the cap member 20 is removed by first rotating the cap member 20 and lancet needle cover member 43 to separate the needle cover member 43 from the lancet main body 41, then axially separating the cap member 20 from the housing 11. The distal portion of the trigger nose 41 extending from the housing neck member 15 is now exposed, and the lancet main body 41 is prevented by the trigger cocking flanges 55 from moving in the distal direction. The needle 42 is now uncovered, but resides well within housing 11. In this neutral or pre-cocked condition the trigger indicator panel 58 is positioned forward of the housing status window 19, such that the lancet 40 can be seen through the status window. Preferably, the lancet 40 and the indicator panel 58 are of different colors for easier indication of the status of the lancet assembly.

To cock and fire the lancet assembly, the user grasps the housing 11 and presses the exposed trigger nose member 51 against the skin, in effect advancing the housing 11 relative to the trigger member 50, or alternatively presses the skin surface against the nose member 51. Thus contact with the skin surface pushes the trigger member 50 into the housing in the proximal direction. As the trigger member 50 moves rearward, the trigger cocking flanges 55 abutting the lancet cocking lugs 48 cause the lancet main body 41 to move rearward as well. The rearward movement of the lancet body 41 compresses and loads the spring member 60. Simultaneously, the arm detention members 56 move along the angled guide surface members 38, causing the arm members 54 to flex and spread outwardly, the angled guide surface members 38 acting in the nature of cam surfaces and the arm detention members 56 acting in the nature of cam followers. As the arm members 54 are spread apart, the distance between the trigger cocking flanges 55 increases until the interior edges of the cocking flanges 55 are farther apart than the exterior edges of the lancet cocking lugs 48 and no longer prevent movement of the lancet main body 41 in the distal direction. The compressed spring 60 is now released and propels the lancet main body 41 and the needle 42 axially such that the needle 42 briefly extends beyond the distal end 12 of the housing neck member 15 and beyond the distal end of the rearwardly disposed trigger nose member 51, thereby piercing the skin of the user, as shown in FIG. 18.

The spring 60 now retracts into its neutral position, thereby drawing the lancet main body 41 and the needle 42 completely back into the housing 11, as shown in FIG. 19. Rearward movement of the trigger member 50 relative to the housing 11 during the cocking operation has positioned the trigger arm detention members 56 to the rear and beyond the proximal ends of the angled guide surfaces 38, thereby allowing the biased arm members 54 to rebound toward the center or move inwardly back toward their neutral position. The detention members 56 now abut the proximal side of detention shoulders 39, the catch members 57 insuring that the detention shoulders 39 securely retain the detention members 56 and free ends of the trigger arms 54, such that the trigger member 50 is precluded from forward movement. The trigger cocking flanges 55 remain disposed to the rear of the lancet cocking lugs 48 and thus cannot be used to move the lancet main body 41 in the proximal direction to re-compress the spring 60 even if the trigger nose 51 is pushed into the housing 11. The trigger member 40 is now restrained or locked in a retracted position relative to the housing 11, and the indicator panel 58 is now positioned beneath the housing status window 19, thereby providing a visible indication that the device has been used. Even without replacement of the cap member 20, the needle 42 remains fully recessed within the housing 11 to preclude accidental contact and there is no operational mechanism that now allows the needle 42 to be advanced so as to extend from the housing 11.

It is understood that equivalents and substitutions for certain elements described above may be obvious to those of ordinary skill in the art, and thus the true scope and definition of the invention is to be as set forth in the following claims.

I claim:

1. A lancet assembly comprising:
a housing comprising a proximal end, a distal end, a distal opening, an angled guide surface and a detention shoulder;
a cap removably mounted onto said housing and covering said distal opening;
a trigger member positioned within said housing, said trigger member comprising a nose extending through said distal opening of said housing, a body, a lancet bore extending through said nose and said body, an arm member connected to said body and extending toward said proximal end of said housing, said arm member having a free end, a detention member mounted on said free end of said arm member, and a cocking flange positioned on said arm member;
a lancet positioned within said housing and extendable through said lancet bore of said trigger member and through said distal opening of said housing, said lancet comprising a main body, a needle extending from said main body, and a cocking lug;
a spring member connected to said lancet;
wherein said trigger member and said lancet are retained within said housing in a manner allowing movement of said trigger member toward said proximal end of said housing and allowing movement of said lancet toward said proximal end and said distal end of said housing;
wherein in a pre-cocked condition said needle is positioned within said housing, and said cocking flange of said trigger member is positioned closer to said distal end of said housing than said cocking lug of said lancet;
wherein with said cap removed from said housing said trigger member may be moved toward said proximal end of said housing such that said cocking flange abuts said cocking lug, thereby moving said lancet main body toward said proximal end of said housing and loading said spring, such that said free end of said arm member is biased outwardly by said angled guide surface as said trigger member is moved toward said proximal end of said housing, such that said cocking flange is moved out of contact with said cocking lug, thereby allowing said spring to propel said lancet needle through and beyond said distal opening of said housing, and such that said detention member of said arm member is restrained by said detention shoulder of said angled guide surface, thereby preventing movement of said trigger member toward said distal end of said housing;
wherein after extension of said needle beyond said housing distal opening said spring retracts said needle into said housing, said cocking lug of said lancet now being positioned closer to the distal end of said housing than said cocking flange of said trigger member, such that said trigger member is now incapable of moving said lancet axially toward said proximal end of said housing.

2. The lancet assembly of claim 1, wherein said housing comprises a pair of angled guide surfaces and detention shoulders, and wherein said trigger member comprises a pair of arm members and detention members.

3. The lancet assembly of claim 2, wherein said lancet comprises a pair of cocking lugs and said trigger member comprises a pair of cocking flanges.

4. The lancet assembly of claim 1, further comprising a needle cover removably connected to said lancet main body and covering said needle, wherein said lancet needle cover is retained by said cap such that removal of said cap from said housing results in removal of said lancet needle cover from said lancet main body.

5. A lancet assembly comprising:
a housing comprising a proximal end, a distal end, a distal opening, an angled guide surface and a detention shoulder;
a cap removably mounted onto said housing and covering said distal opening;
a lancet retained within said housing, said lancet comprising a main body, a needle extending from said main body, and a cocking lug, said lancet comprising a longitudinal axis, wherein said lancet is movable in both axial directions relative to said housing;
a trigger member positioned within said housing, said trigger member comprising a nose extending through said distal opening of said housing, a body, a lancet bore extending through said nose and said body, an arm member connected to said body and extending toward said proximal end of said housing, said arm member having a free end, a detention member mounted on said free end of said arm member, and a cocking flange positioned on said arm member, wherein said trigger member is movable in the axial direction toward said proximal end of said housing;
said lancet being disposed within said lancet bore of said trigger member;
a spring member connected to said lancet;
wherein in a pre-cocked condition said needle is positioned within said housing, and said cocking flange of said trigger member is positioned closer to said distal end of said housing than said cocking lug of said lancet;
wherein with said cap removed from said housing said trigger member may be moved axially toward said proximal end of said housing such that said cocking flange abuts said cocking lug, thereby moving said lancet main body axially toward said proximal end of said housing and loading said spring, such that said free end of said arm member is biased outwardly by said angled guide surface as said trigger member is moved toward said proximal end of said housing, such that said cocking flange is moved out of contact with said cocking lug, thereby allowing said spring to propel said lancet needle axially through and beyond said distal opening of said housing, and such that said detention member of said arm member is restrained by said detention shoulder of said angled guide surface, thereby preventing axial movement of said trigger member toward said distal end of said housing;
wherein after extension of said needle beyond said housing distal opening said spring axially retracts said needle into said housing, said cocking lug of said lancet now being positioned closer to the distal end of said housing than said cocking flange of said trigger member, such that said trigger member is rendered incapable of moving said lancet in the axial direction toward said proximal end of said housing.

6. The lancet assembly of claim 5, wherein said housing comprises a pair of angled guide surfaces and detention shoulders, and wherein said trigger member comprises a pair of arm members and detention members.

7. The lancet assembly of claim 6, wherein said lancet comprises a pair of cocking lugs and said trigger member comprises a pair of cocking flanges.

8. The lancet assembly of claim 5, further comprising a needle cover removably connected to said main body and covering said needle, wherein said lancet needle cover is retained by said cap such that removal of said cap from said housing results in removal of said lancet needle cover from said lancet main body.

9. A lancet assembly comprising:
a housing comprising a proximal end, a distal end, a distal opening, a pair of angled guide surfaces and a pair of detention shoulders;
a cap removably mounted onto said housing and covering said distal opening;
a trigger member positioned within said housing, said trigger member comprising a nose extending through said distal opening of said housing, a body, a lancet bore extending through said nose and said body, a pair of arm members connected to said body and extending toward said proximal end of said housing, said arm members each having a free end, a detention member mounted on each said free end of said arm members, and a cocking flange positioned on each said arm member;
a lancet positioned within said housing and extending through said lancet bore, said lancet comprising a main body, a needle extending from said main body, and a pair of cocking lugs; and
a spring member connected to said lancet and to said housing.

10. The lancet assembly of claim 9, wherein said detention members abut said angled guide surfaces and said cocking flanges abut said cocking lugs.

11. The lancet assembly of claim 9, further comprising a needle cover removably connected to said lancet main body and covering said needle.

12. The lancet assembly of claim 11, wherein said needle cover is connected to said cap in a manner such that rotating said cap relative to said housing rotates said needle cover and separates said needle cover from said lancet main body.

13. The lancet assembly of claim 9, wherein said trigger member and said lancet are disposed within said housing such that said trigger member is movable toward the proximal end of said housing and said lancet is movable toward said proximal end and said distal end of said housing.

14. The lancet assembly of claim 13, wherein movement of said trigger member toward said proximal end of said housing results in movement of said lancet first toward proximal end of said housing and then toward said distal end.

15. The lancet assembly of claim 14, wherein after said lancet moves toward said distal end of said housing, said spring retracts said lancet needle into said housing by moving said lancet toward the proximal end of said housing.

16. The lancet assembly of claim 9, wherein said trigger member, said cocking flanges, said cocking lugs, said angled guide surfaces and said arm members are disposed within said housing such that movement of said trigger member toward said proximal end of said housing results in separation of said arm members such that said cocking flanges no longer abut said cocking lugs, thereby allowing said lancet to move toward said distal end of said housing.

17. The lancet assembly of claim 16, wherein said detention members and said detention shoulders are disposed within said housing such that said movement of said trigger member toward said proximal end of said housing results in said detention members abutting said detention shoulders, thereby preventing said trigger member from moving toward said distal end of said housing.

18. The lancet assembly of claim 15, wherein said trigger member, said cocking flanges, said cocking lugs, said angled guide surfaces and said arm members are disposed within said housing such that said movement of said trigger member toward said proximal end of said housing results in separation of said arm members such that said cocking flanges no longer abut said cocking lugs, thereby allowing said lancet to move toward said distal end of said housing.

19. The lancet assembly of claim 18, wherein said detention members and said detention shoulders are disposed within said housing such that said movement of said trigger member toward said proximal end of said housing results in said detention members abutting said detention shoulders, thereby preventing said trigger member from moving toward said distal end of said housing.
